(19) United States
(12) Patent Application Publication
(10) Pub. No.: US 2025/0308031 A1
(43) Pub. Date: Oct. 2, 2025

(54) MEDICAL IMAGE PROCESSING DEVICE, MEDICAL IMAGE PROCESSING METHOD, AND PROGRAM

(71) Applicant: FUJIFILM Corporation, Tokyo (JP)

(72) Inventor: Yusuke SUGIMOTO, Tokyo (JP)

(73) Assignee: FUJIFILM Corporation, Tokyo (JP)

(21) Appl. No.: 19/234,228

(22) Filed: Jun. 10, 2025

Related U.S. Application Data
(63) Continuation of application No. PCT/JP2023/043594, filed on Dec. 6, 2023.

(30) Foreign Application Priority Data
Dec. 12, 2022 (JP) 2022-198135

(51) Int. Cl. G06T 7/11; A61B 5/00; A61B 5/055; A61B 6/03; A61B 6/46; G06T 7/00; G06T 11/00

(52) U.S. Cl. CPC G06T 7/11; A61B 5/055; A61B 5/7435; A61B 6/032; A61B 6/465; A61B 6/466; G06T 7/0016; G06T 11/003; G06T 2200/04; G06T 2200/24; G06T 2207/10081; G06T 2207/10088; G06T 2207/20104

(57) ABSTRACT

Provided are a medical image processing device, a medical image processing method, and a program that reduce a load on a user in a case of designating a three-dimensional region and improve an interpretation accuracy and efficiency of the three-dimensional region. A first image, which is a tomographic image or cross-sectional image orthogonal to a first direction of three-dimensional medical image data, is displayed on a display device, a rectangular region designated in the displayed first image is received, a length of a normal line of the first image is determined based on the rectangular region, a three-dimensional region surrounded by a rectangular parallelepiped defined by the rectangular region and the normal line in the three-dimensional medical image data is determined, image processing is performed on the determined three-dimensional region, and a result of the image processing is displayed on the display device.

### CROSS-REFERENCE TO RELATED APPLICATIONS

The present application is a Continuation of PCT International Application No. PCT/JP2023/043594 filed on Dec. 6, 2023 claiming priority under 35 U.S.C § 119(a) to Japanese Patent Application No. 2022-198135 filed on Dec. 12, 2022. Each of the above applications is hereby expressly incorporated by reference, in its entirety, into the present application.

### BACKGROUND OF THE INVENTION

### 1. Field of the Invention

The present invention relates to a medical image processing device, a medical image processing method, and a program, and particularly to a technology for designating a three-dimensional region of a three-dimensional image.

### 2. Description of the Related Art

In a case where image processing is performed by designating a three-dimensional region on a viewer that displays a designated tomographic image of a plurality of tomographic images imaged by a computed tomography (CT) device, a magnetic resonance imaging (MRI) device, or the like, it takes time and effort to designate a corresponding region in each tomographic image.

In order to solve such a problem, JP2013-180153A proposes a method of calculating a height in a normal direction to acquire a rectangular parallelepiped of a region of interest in a case where two end points on a tomographic image are designated by a user operation.

### SUMMARY OF THE INVENTION

However, the method disclosed in JP2013-180153A specifies a corresponding tomographic image based on the amount of deformation of a plurality of positions included in the region of interest, and does not provide information on the region of interest.

The present invention has been made in view of such circumstances, and an object of the present invention is to provide a medical image processing device, a medical image processing method, and a program that reduce a load on a user in a case of designating a three-dimensional region and improve an interpretation accuracy and efficiency.

In order to achieve the above object, a medical image processing device according to a first aspect of the present disclosure comprises at least one processor; and at least one memory that stores a command to be executed by the at least one processor, in which the at least one processor displays, on a display device, a first image, which is a tomographic image or cross-sectional image orthogonal to a first direction of three-dimensional medical image data, receives a rectangular region designated in the displayed first image, determines a length of a normal line of the first image based on the rectangular region, determines a three-dimensional region surrounded by a rectangular parallelepiped defined by the rectangular region and the normal line in the three-dimensional medical image data, performs image processing on the determined three-dimensional region, and displays, on the display device, a result of the image processing.

According to the aspect, the load on the user in a case of designating the three-dimensional region can be reduced and the interpretation accuracy and efficiency of the three-dimensional region can be improved.

In the medical image processing device according to a second aspect of the present disclosure, in the medical image processing device according to the first aspect, it is preferable that the at least one processor receives a modified length of the normal line, modifies the three-dimensional region with the modified length of the normal line, and performs the image processing on the modified three-dimensional region. As a result, the result of the image processing of the three-dimensional region modified to an appropriate size can be displayed.

In the medical image processing device according to a third aspect of the present disclosure, in the medical image processing device according to the second aspect, it is preferable that the at least one processor receives designation of enlargement and/or reduction of the designated rectangular region, and modifies the length of the normal line in conjunction with the designation of enlargement and/or reduction. As a result, a size of the three-dimensional region can be modified by modifying a size of the rectangular region.

In the medical image processing device according to a fourth aspect of the present disclosure, in the medical image processing device according to the first aspect, it is preferable that the at least one processor receives the rectangular region having a square shape, and determines the length of the normal line to be a length of one side of the square shape. As a result, the length of the normal line can be determined to an appropriate length.

In the medical image processing device according to a fifth aspect of the present disclosure, in the medical image processing device according to the first aspect, it is preferable that the at least one processor displays the first image and the result of the image processing side by side. As a result, the efficiency of the interpretation can be improved.

In the medical image processing device according to a sixth aspect of the present disclosure, in the medical image processing device according to the fifth aspect, it is preferable that the at least one processor performs the image processing on a past three-dimensional region corresponding to the determined three-dimensional region in past three-dimensional medical image data corresponding to the three-dimensional medical image data, and displays a result of the image processing on the past three-dimensional region further side by side. As a result, the efficiency of the interpretation can be improved.

In the medical image processing device according to a seventh aspect of the present disclosure, in the medical image processing device according to the first aspect, it is preferable that the at least one processor displays, on the display device, a slider bar indicating a position of the first image in the three-dimensional medical image data in the first direction, and displays, on the slider bar, a range corresponding to the normal line. As a result, the user can check the range of the normal line.

In the medical image processing device according to an eighth aspect of the present disclosure, in the medical image processing device according to the first aspect, it is preferable that the at least one processor displays, on the display device, a numerical value indicating a range corresponding to the normal line in the first direction of the three-dimensional medical image data. As a result, the user can check the range of the normal line.

In the medical image processing device according to a ninth aspect of the present disclosure, in the medical image processing device according to the first aspect, it is preferable that the at least one processor defines a rectangular parallelepiped in which the rectangular region is located at a center in the first direction. As a result, the three-dimensional region can be determined at an appropriate position.

In the medical image processing device according to a tenth aspect of the present disclosure, in the medical image processing device according to any one of the first to ninth aspects, it is preferable that the image processing includes three-dimensional reconstruction, and the result of the image processing includes at least one of an image of an axial cross section, an image of a sagittal cross section, or an image of a coronal cross section. As a result, the accuracy of the interpretation can be improved.

In order to achieve the above object, a medical image processing method according to an eleventh aspect of the present disclosure comprises causing at least one processor to: display, on a display device, a first image, which is a tomographic image or cross-sectional image orthogonal to a first direction of three-dimensional medical image data, receive a rectangular region designated in the displayed first image, determine a length of a normal line of the first image based on the rectangular region, determine a three-dimensional region surrounded by a rectangular parallelepiped defined by the rectangular region and the normal line in the three-dimensional medical image data, perform image processing on the determined three-dimensional region, and display, on the display device, a result of the image processing.

According to the aspect, the load on the user in a case of designating the three-dimensional region can be reduced and the interpretation accuracy and efficiency of the three-dimensional region can be improved.

In order to achieve the above object, a program according to a twelfth aspect of the present disclosure is a program for causing a computer to execute the medical image processing method according to the eleventh aspect. The present disclosure also includes a non-transitory computer-readable recording medium, such as a compact disk-read only memory (CD-ROM), on which the program according to the twelfth aspect is recorded.

According to the invention, the load on the user in a case of designating the three-dimensional region can be reduced and the interpretation accuracy and efficiency of the three-dimensional region can be improved.

### BRIEF DESCRIPTION OF THE DRAWINGS

FIG. 1 is an overall configuration diagram of a medical image processing system.

FIG. 2 is a block diagram showing an electric configuration of a medical image processing device.

FIG. 3 is a block diagram showing a functional configuration of the medical image processing device.

FIG. 4 is a flowchart showing a medical image processing method.

FIG. 5 is a view showing an example of three-dimensional image data consisting of a plurality of tomographic images.

FIG. 6 is a view showing an example of a displayed tomographic image.

FIG. 7 is a view showing an example of a rectangular region designated in a first image.

FIG. 8 is a view showing an example of a normal line determined in the first image.

FIG. 9 is a view showing an example of a three-dimensional region surrounded by a rectangular parallelepiped.

FIG. 10 is a view showing an example of a three-dimensional region surrounded by a rectangular parallelepiped.

FIG. 11 is a view showing an example of a displayed image processing result.

FIG. 12 is a view showing another example of the displayed image processing result.

### DESCRIPTION OF THE PREFERRED EMBODIMENTS

Hereinafter, preferable embodiments of the present invention will be described in detail with reference to the accompanying drawings. Here, a medical image processing system will be described as an example of a medical image processing device, a medical image processing method, and a program according to the embodiment of the present invention.

### Medical Image Processing System

The medical image processing system according to the present embodiment is a system that determines a three-dimensional region including a region of interest of three-dimensional medical image data from a displayed two-dimensional tomographic image or cross-sectional image, performs image processing on the determined three-dimensional region, and displays a processing result. The doctor acquires information regarding the region of interest from the image processing result of the displayed three-dimensional region, and creates an interpretation report. As a result, the load on the user in a case of designating the three-dimensional region is reduced, and the interpretation accuracy and efficiency are improved.

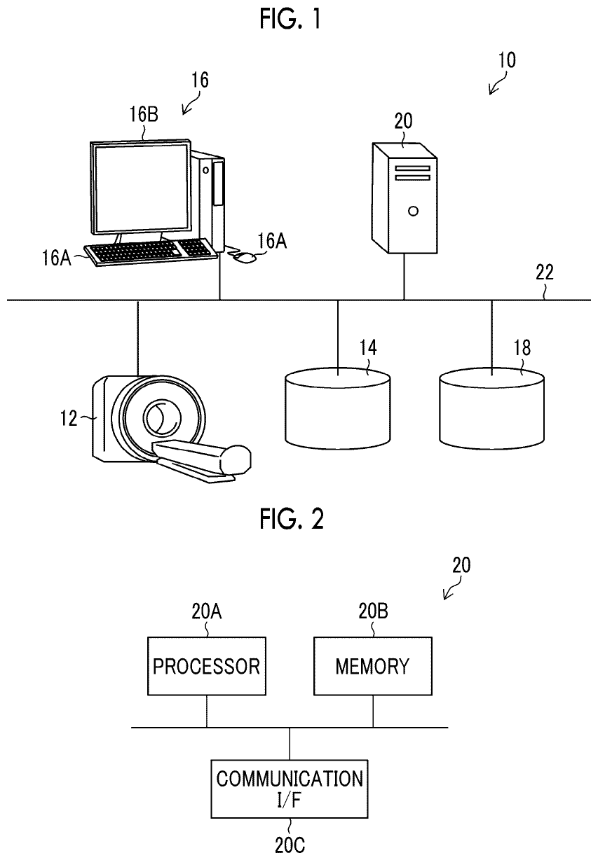

FIG. 1 is an overall configuration diagram of a medical image processing system 10. As shown in FIG. 1, the medical image processing system 10 comprises a medical image examination device 12, a medical image database 14, a user terminal device 16, an interpretation report database 18, and a medical image processing device 20.

The medical image examination device 12, the medical image database 14, the user terminal device 16, the interpretation report database 18, and the medical image processing device 20 are connected via a network 22 to transmit and receive data to and from each other. The network 22 includes a wired or wireless local area network (LAN) for communication and connection of various devices in a medical institution. The network 22 may include a wide area network (WAN) for connecting LANs of a plurality of medical institutions to each other.

The medical image examination device 12 is an imaging device that images an examination target part of a subject and generates a medical image. Examples of the medical image examination device 12 include an X-ray imaging device, a computed tomography (CT) device, a magnetic resonance imaging (MRI) device, a positron emission tomography (PET) device, an ultrasound device, a computed radiography (CR) device using a planar X-ray detector, and an endoscope device.

The medical image database 14 is a database for managing medical images imaged by the medical image examination device 12. As the medical image database 14, a computer comprising a large-capacity storage device for storing the medical image is applied. The computer incorporates software that provides a function of a database management system.

The medical image may be a plurality of tomographic images imaged by the CT device, the MRI device, or the like, or may be a three-dimensional reconstructed image reconstructed by using the plurality of tomographic images. The medical image may be a cross-sectional image in any direction of the three-dimensional reconstructed image.

As a format of the medical image, digital imaging and communications in medicine (Dicom) standards can be applied. The medical image may be added with accessory information (Dicom tag information) defined in the Dicom standards. The term “image” in the present specification includes not only a meaning of the image itself, such as a photograph, but also a meaning of image data that is a signal indicating the image.

The user terminal device 16 is a terminal device for the doctor to create and view the interpretation report, and includes viewer software for the doctor to view the medical image. As the user terminal device 16, for example, a personal computer is applied. The user terminal device 16 may be a workstation, or may be a tablet terminal. The user terminal device 16 comprises an input device 16A and a display 16B which is a display device. The input device 16A may include a mouse and a keyboard. The doctor uses the input device 16A to input an instruction to display the medical image. The user terminal device 16 displays the medical image on the display 16B. In addition, the user terminal device 16 displays a result of image processing, which will be described later, in the medical image processing device 20 on the display 16B. The doctor creates the interpretation report by inputting a comment on findings, which is an interpretation result of the medical image, using the input device 16A based on the medical image and the image processing result displayed on the display 16B.

The interpretation report database 18 is a database for managing the interpretation report generated by the doctor in the user terminal device 16. The interpretation report may include the image processing result in the medical image processing device 20. As the interpretation report database 18, a computer comprising a large-capacity storage device for storing the interpretation report is applied. The computer incorporates software that provides a function of a database management system. The medical image database 14 and the interpretation report database 18 may be configured with one computer.

The medical image processing device 20 is a device that performs the image processing on a designated region of interest of the medical image. As the medical image processing device 20, a personal computer or a workstation (an example of “computer”) can be applied. FIG. 2 is a block diagram showing an electric configuration of the medical image processing device 20. As shown in FIG. 2, the medical image processing device 20 comprises a processor 20A, a memory 20B, and a communication interface 20C.

The processor 20A executes a command stored in the memory 20B. A hardware structure of the processor 20A includes the following various processors. The various processors include a central processing unit (CPU) that is a general-purpose processor operating as various functional units by executing software (a program), a graphics processing unit (GPU) that is a processor specialized in image processing, a programmable logic device (PLD) such as a field programmable gate array (FPGA) that is a processor having a circuit configuration changeable after manufacture, a dedicated electric circuit such as an application specific integrated circuit (ASIC) that is a processor having a circuit configuration dedicatedly designed to execute specific processing, and the like.

One processing unit may be configured with one processor among these various processors, or may be configured with two or more same or different kinds of processors (for example, a combination of a plurality of FPGAs, a combination of the CPU and the FPGA, or a combination of the CPU and the GPU). A plurality of functional units may be configured with one processor. As an example of the plurality of functional units configured with one processor, first, as represented by a computer such as a client or a server, there is a form in which one processor is configured with a combination of one or more CPUs and software and the processor operates as a plurality of functional units. Second, as represented by a system on chip (SoC) and the like, there is a form using a processor that implements functions of the whole system including a plurality of functional units in one integrated circuit (IC) chip. Accordingly, various functional units are configured using one or more of the various processors as a hardware structure.

Furthermore, the hardware structure of the various processors is more specifically an electric circuit (circuitry) in which circuit elements, such as semiconductor elements, are combined.

The memory 20B stores the command to be executed by the processor 20A. The memory 20B includes a random access memory (RAM) and a read only memory (ROM) (not illustrated). The processor 20A uses the RAM as a work area, executes software using various parameters and programs including a medical image processing program described later, which are stored in the ROM, and executes various kinds of processing of the medical image processing device 20 by using the parameters stored in the ROM, and the like.

The communication interface 20C controls communication with the medical image examination device 12, the medical image database 14, the user terminal device 16, and the interpretation report database 18 via the network 22, in accordance with a predetermined protocol.

The medical image processing device 20 may be a cloud server accessible from a plurality of medical institutions via the Internet. The processing performed by the medical image processing device 20 may be a cloud service based on a charge system or a fixed fee system.

### Functional Configuration of Medical Image Processing Device

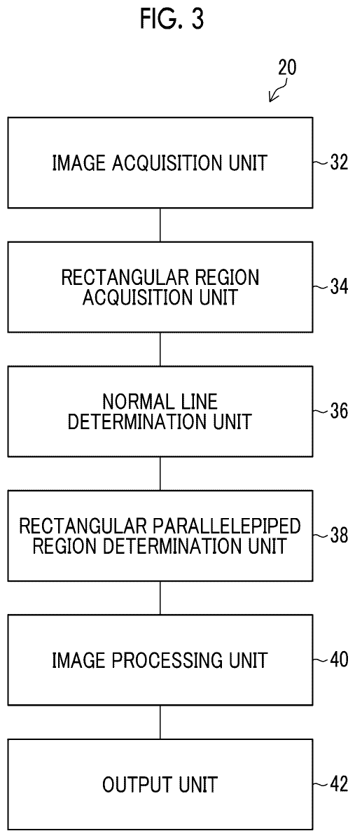

FIG. 3 is a block diagram showing a functional configuration of the medical image processing device 20. Each function of the medical image processing device 20 is realized by the processor 20A executing the medical image processing program stored in the memory 20B. As shown in FIG. 3, the medical image processing device 20 comprises an image acquisition unit 32, a rectangular region acquisition unit 34, a normal line determination unit 36, a rectangular parallelepiped region determination unit 38, an image processing unit 40, and an output unit 42.

The output unit 42 outputs various types of data to the user terminal device 16 and displays the various types of data on the display 16B.

The image acquisition unit 32 acquires a three-dimensional medical image (an example of the “three-dimensional medical image data”) from the medical image database 14 and displays a two-dimensional medical image (an example of a “first image”) on the display 16B via the output unit 42. The image acquisition unit 32 may acquire a plurality of tomographic images imaged at a constant slice interval and a constant slice thickness along a body axis direction of the subject by the medical image examination device 12 as the three-dimensional medical image, and display any tomographic image among the plurality of tomographic images as the two-dimensional medical image. The doctor can display a tomographic image including a region of interest, such as a lesion, by selecting a tomographic image at a desired slice position among the plurality of tomographic images using the input device 16A.

The image acquisition unit 32 may acquire a plurality of high-resolution virtual thin slice tomographic images having a relatively small slice interval and slice thickness as the three-dimensional medical image. The thin slice tomographic images are generated from a plurality of low-resolution thick slice tomographic images having a relatively large slice interval and slice thickness, which are imaged by the medical image examination device 12 as a three-dimensional medical image. The image acquisition unit 32 may acquire the thin slice tomographic image from the medical image database 14, or may acquire the thick slice tomographic image from the medical image database 14 and generate the thin slice tomographic image. The doctor can display a thin slice tomographic image including a region of interest as the two-dimensional medical image by selecting a tomographic image at a desired slice position among the plurality of thin slice tomographic images using the input device 16A.

The image acquisition unit 32 may acquire the three-dimensional reconstructed image reconstructed using the plurality of tomographic images as the three-dimensional medical image, and display a cross-sectional image obtained by cutting out a cross section orthogonal to any direction of the three-dimensional reconstructed image as the two-dimensional medical image. The doctor can display the cross-sectional image including a region of interest by selecting a cross section direction and a cross section position of the cross section image to be displayed using the input device 16A.

The image acquisition unit 32 may acquire a medical image in which the same subject is imaged by the medical image examination device 12 in the past as the three-dimensional medical image, and display a two-dimensional medical image based on the medical image imaged in the past as the two-dimensional medical image.

Here, the image acquisition unit 32 displays the first image, which is a tomographic image or cross-sectional image orthogonal to a first direction, on the display 16B. The first direction is, for example, the body axis direction of the subject.

The rectangular region acquisition unit 34 receives and acquires a rectangular region designated in the first image displayed on the display 16B from the user terminal device 16. The doctor designates the rectangular region including a region of interest on the displayed first image using the input device 16A. The doctor may enlarge and/or reduce the size of the rectangular region using the input device 16A.

The normal line determination unit 36 determines a length of the normal line of the displayed first image based on the rectangular region acquired by the rectangular region acquisition unit 34. The normal line of the first image is a straight line parallel to the first direction. In a case where the rectangular region acquisition unit 34 acquires a rectangular region having a square shape, the normal line determination unit 36 may determine the length of the normal line to be the same as a length of one side of the square shape. In a case where the rectangular region acquisition unit 34 receives designation of enlargement and/or reduction of the rectangular region, the normal line determination unit 36 may modify the length of the normal line in conjunction with the designation of enlargement and/or reduction.

The rectangular parallelepiped region determination unit 38 determines a three-dimensional region included in the three-dimensional medical image, the three-dimensional region being surrounded by a rectangular parallelepiped defined by the rectangular region acquired by the rectangular region acquisition unit 34 and the normal line determined by the normal line determination unit 36. The rectangular parallelepiped region determination unit 38 may define a rectangular parallelepiped in which the rectangular region is located at the center in the first direction.

The rectangular parallelepiped region determination unit 38 calculates a range corresponding to the normal line in the three-dimensional medical image including the first image based on the length of the normal line determined by the normal line determination unit 36. The rectangular parallelepiped region determination unit 38 may display the calculated range on a slider bar to be described later in a visible manner, or may display a numerical value indicating the calculated range in an overlay manner on the first image displayed on the display 16B.

The image processing unit 40 performs the image processing on the three-dimensional region determined by the rectangular parallelepiped region determination unit 38. The image processing unit 40 may generate a two-dimensional or three-dimensional image by reconstructing the three-dimensional region. The image processing unit 40 outputs an image processing result to the user terminal device 16 via the output unit 42 and displays the image processing result on the display 16B. The image processing unit 40 may display the first image and the image processing result side by side.

### Medical Image Processing Method

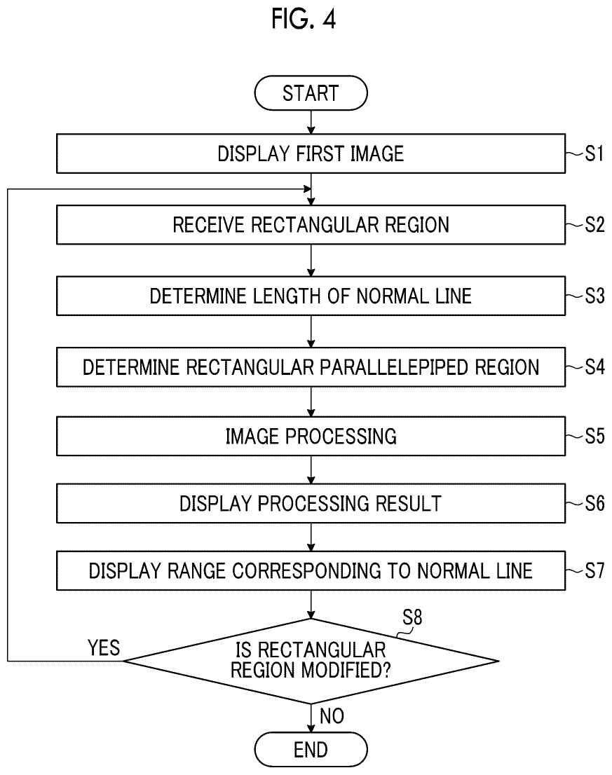

FIG. 4 is a flowchart showing the medical image processing method using the medical image processing device 20. The medical image processing method is a method of determining the three-dimensional region including a region of interest of the three-dimensional medical image from the displayed two-dimensional tomographic image or cross-sectional image, and performing the image processing on the determined three-dimensional region to display a processing result. The medical image processing method is implemented by the processor 20A executing the medical image processing program stored in the memory 20B. The medical image processing program may be provided by a computer-readable non-transitory storage medium, or may be provided via the Internet.

In step S1, the doctor activates the viewer software of the user terminal device 16 and selects an examination of the subject to be interpreted. The image acquisition unit 32 acquires the medical image imaged in the selected examination from the medical image database 14. In addition, the image acquisition unit 32 displays the first image based on the medical image on the display 16B via the output unit 42. For example, the image acquisition unit 32 acquires a three-dimensional image consisting of a plurality of tomographic images and displays any tomographic image among the plurality of tomographic images. The image acquisition unit 32 may acquire a three-dimensional reconstructed image and display a cross-sectional image obtained by cutting out a cross section orthogonal to any direction of the three-dimensional reconstructed image.

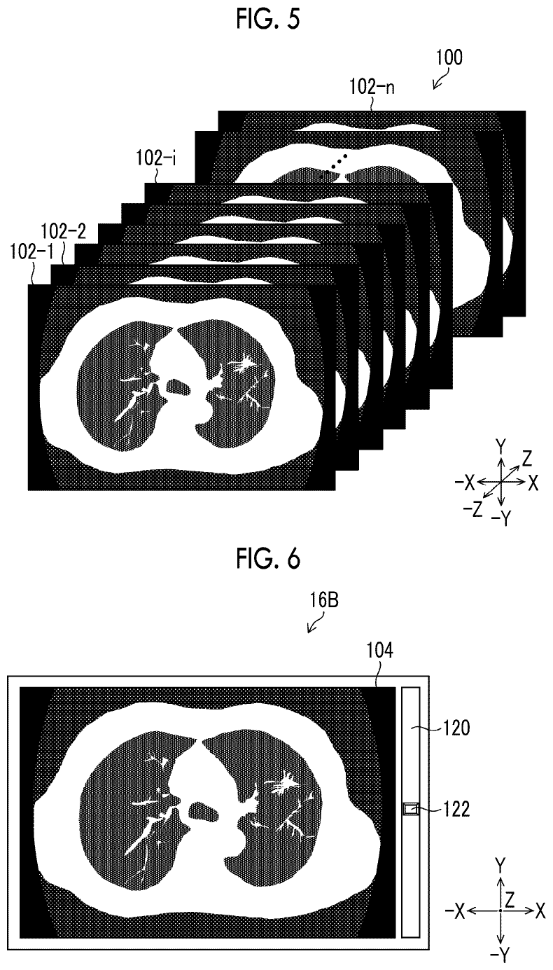

FIG. 5 is a view showing an example of the three-dimensional image consisting of the plurality of tomographic images. A three-dimensional image 100 includes a plurality of tomographic images 102-1, 102-2, . . . , 102-i, . . . , 102-n that are orthogonal to a Z direction which is the body axis direction. It is noted that n is a natural number, and i is a natural number smaller than n. The plurality of tomographic images 102-1 to 102-n are images having slice planes parallel to an XY-plane imaged at a constant slice interval and a constant slice thickness along the Z direction. The image acquisition unit 32 displays the slice plane of any tomographic image among the plurality of tomographic images 102-1 to 102-n on the display 16B in response to an input from the input device 16A.

FIG. 6 is a view showing an example of the displayed tomographic image. In FIG. 6, a tomographic image 104 displayed on the display 16B is a tomographic image designated by the doctor among the plurality of tomographic images 102-1 to 102-n. A slider bar 120 is displayed on the display 16B together with the tomographic image 104. The slider bar 120 indicates a range of the three-dimensional image 100 in the Z direction. For example, an upper end of the slider bar 120 indicates a slice position of the tomographic image 102-1, and a lower end of the slider bar 120 indicates a slice position of the tomographic image 102-n. A slider 122 indicating a slice position of the tomographic image 104 displayed on the display 16B is provided on the slider bar 120. The doctor can change the slice position of the three-dimensional image by changing a position of the slider 122 using the input device 16A, and display the tomographic image 104 at a desired slice position on the display 16B.

In step S2, the doctor designates the region of interest of the first image displayed on the display 16B as a two-dimensional rectangular region using the input device 16A. The rectangular region acquisition unit 34 receives and acquires the rectangular region designated in the first image from the user terminal device 16.

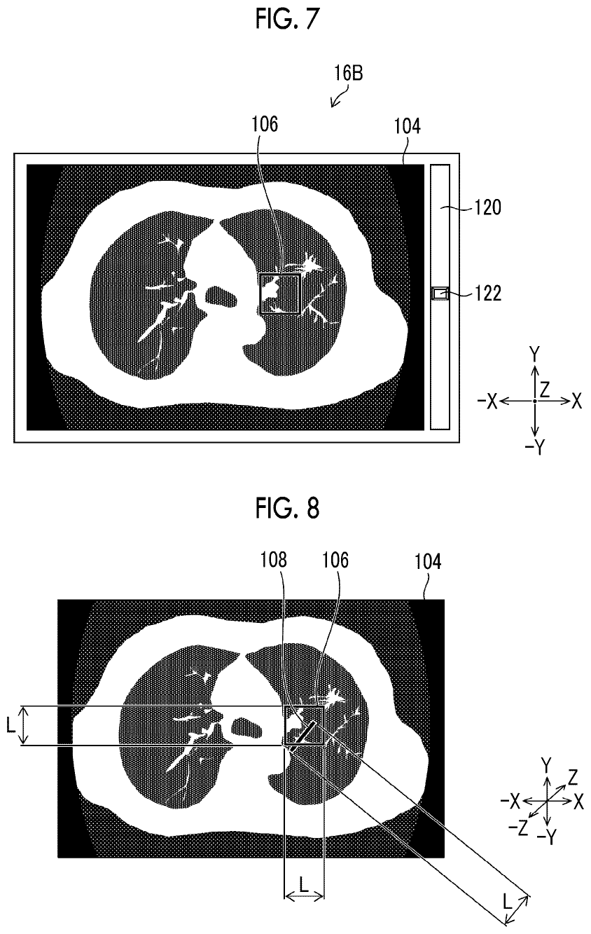

FIG. 7 is a view showing an example of the rectangular region designated in the first image. Here, a rectangular region 106 is designated in the tomographic image 104. The rectangular region 106 is a region designated in the tomographic image 104 by the doctor using the input device 16A and is, for example, a region surrounding a region of interest captured in the tomographic image 104. Here, the rectangular region 106 is a square region having a side length of L.

In step S3, the normal line determination unit 36 determines the length of the normal line (a line perpendicular to the XY plane) of the first image displayed on the display 16B based on the rectangular region acquired in step S2. The normal line determination unit 36 may determine the length of the normal line in a front direction (-Z direction) and the length of the normal line in a back direction (+Z direction), respectively.

FIG. 8 is a view showing an example of the normal line determined in the first image. Here, a normal line 108 of the length Lis determined in the tomographic image 104. That is, the length of the normal line 108 is equal to the length of one side of the rectangular region 106 having a square shape. The normal line 108 is virtual and is not displayed on the display 16B.

In step S4, the rectangular parallelepiped region determination unit 38 determines a three-dimensional region included in the three-dimensional image and surrounded by a rectangular parallelepiped defined by the rectangular region acquired in step S2 and the normal line determined in step S3.

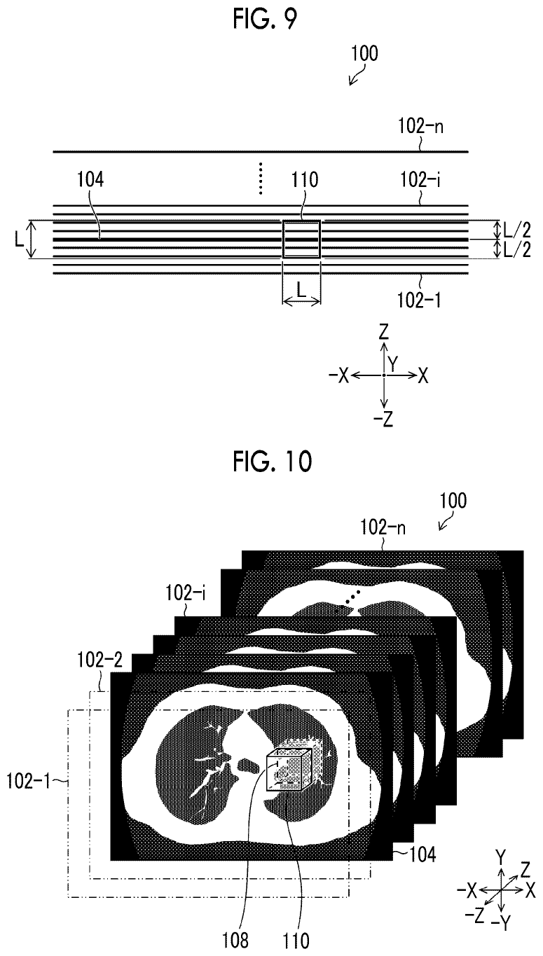

FIGS. 9 and 10 are views showing an example of the three-dimensional region surrounded by the rectangular parallelepiped. FIG. 9 is a view of the three-dimensional image 100 as viewed from a Y direction, and FIG. 10 is a view showing the three-dimensional image 100 in the same manner as FIG. 5. As shown in FIGS. 9 and 10, the rectangular parallelepiped defined by the rectangular region acquired in step S2 and the normal line determined in step S3 is a rectangular parallelepiped of which a top surface is a rectangle obtained by translating the rectangular region 106 in the Z direction by a first length and a bottom surface is a rectangle obtained by translating the rectangular region 106 in the −Z direction by a second length. A sum of the first length and the second length is equal to the length of the normal line. Here, the rectangular parallelepiped region determination unit 38 determines a three-dimensional region 110 surrounded by the rectangular parallelepiped in which the tomographic image 104 having the rectangular region 106 is located at the center in the Z direction. That is, both the first length and the second length are L/2.

As described above, the doctor can easily designate any three-dimensional region from the three-dimensional image by designating the rectangular region in the two-dimensional image.

In step S5, the image processing unit 40 performs the image processing on the three-dimensional region determined in step S4. In addition, in step S6, the image processing unit 40 displays the processing result on the display 16B. Further, in step S7, the rectangular parallelepiped region determination unit 38 displays the range corresponding to the normal line determined in step S3 in the three-dimensional medical image including the first image.

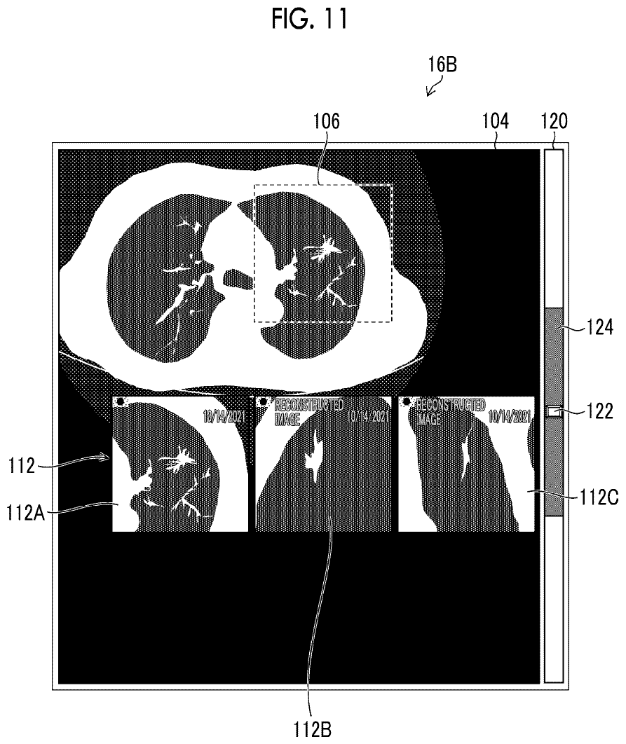

FIG. 11 is a view showing an example of the displayed image processing result. Here, the tomographic image 104 in which the rectangular region 106 is disposed and an image processing result 112 are displayed side by side on the display 16B. The image processing result 112 is a two-dimensional image generated by reconstructing the three-dimensional region 110, and includes an image 112A of an axial cross section, an image 112B of a sagittal cross section, and an image 112C of a coronal cross section. The image 112A of the axial cross section, the image 112B of the sagittal cross section, and the image 112C of the coronal cross section are displayed overlaid on the tomographic image 104. The image processing result 112 only needs to include at least one of the image 112A of the axial cross section, the image 112B of the sagittal cross section, or the image 112C of the coronal cross section.

In addition, as shown in FIG. 11, the slider bar 120 is displayed on the display 16B. A range display unit 124 is provided on the slider bar 120. The range display unit 124 indicates the range corresponding to the normal line 108 in the three-dimensional medical image. Here, the range of the normal line 108 is a range in which the tomographic image 104 is located at the center in the Z direction, so that, in FIG. 11, the range display unit 124 is shown to have the same length above and below the slider 122 as a center.

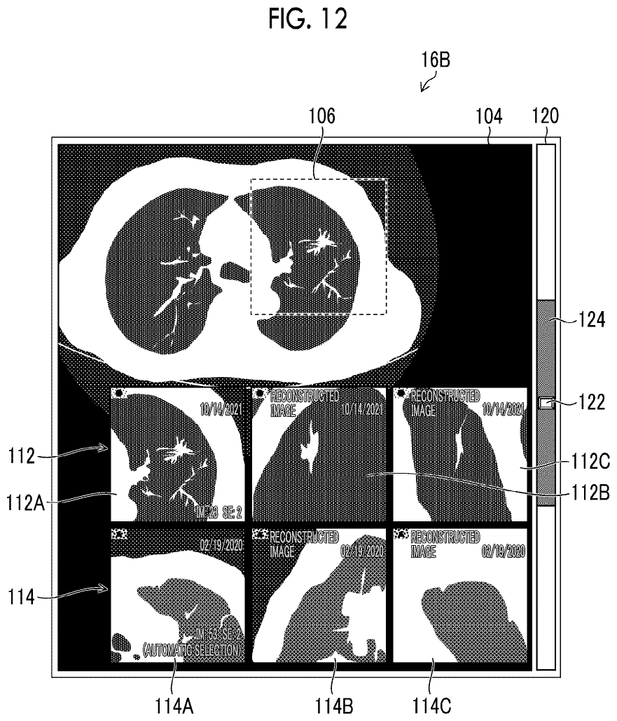

FIG. 12 is a view showing another example of the displayed image processing result. Here, an image processing result 114 is displayed side by side on the display 16B, together with the tomographic image 104 and the image processing result 112. The image processing result 114 is obtained by performing the same image processing on a three-dimensional region of past three-dimensional image data corresponding to the three-dimensional image 100, the three-dimensional region corresponding to the three-dimensional region 110. The past three-dimensional image data corresponding to the three-dimensional image 100 is, for example, a three-dimensional image of the same subject imaged before the imaging of the three-dimensional image 100. The three-dimensional region corresponding to the three-dimensional region 110 is a three-dimensional region of the past three-dimensional image, and is a three-dimensional region in which the same position of the subject as the three-dimensional region of the three-dimensional image 100 is imaged. That is, the region may not be a region of the same coordinate position of an image having the same slice number as the three-dimensional region of the three-dimensional image 100. In order to extract such a three-dimensional region from the past three-dimensional image, registration between the three-dimensional image 100 and the past three-dimensional image is required. The registration between the three-dimensional images may be performed using a known method.

In the example shown in FIG. 12, the image processing result 114 includes an image 114A of an axial cross section, an image 114B of a sagittal cross section, and an image 114C of a coronal cross section, and each of the images is displayed overlaid on the tomographic image 104. The image 114A of the axial cross section, the image 114B of the sagittal cross section, and the image 114C of the coronal cross section are images of cross sections at the same positions as the image 112A of the axial cross section, the image 112B of the sagittal cross section, and the image 112C of the coronal cross section, respectively. As a result, it is possible to compare the current region of interest with the region at the same position in the past. The image processing result 114 only needs to include any one of the image 114A of the axial cross section, the image 114B of the sagittal cross section, or the image 114C of the coronal cross section.

In this way, by displaying the image processing result for the determined three-dimensional region, an interpretation accuracy and efficiency can be improved.

In subsequent step S8, the medical image processing device 20 determines whether the rectangular region has been modified. The doctor can modify a position and a size of the rectangular region 106 using the input device 16A.

In a case where the rectangular region 106 is modified, the process returns to step S2, and the same processing is performed. That is, the modified rectangular region 106 is received (step S2), the length of the normal line 108 corresponding to the modified rectangular region 106 is determined (step S3), the three-dimensional region is determined (step S4), the image processing is performed on the three-dimensional region (step S6), and the processing result is displayed (step S7).

In a case where the doctor performs an operation of enlarging the rectangular region 106 in step S8, the normal line determination unit 36 may modify the length of the normal line 108 to be longer in conjunction with the designation of the enlargement, in step S3. For example, the normal line determination unit 36 may lengthen the length of the normal line 108 according to an enlargement ratio of the rectangular region 106. In addition, in a case where the doctor performs an operation of reducing the rectangular region 106 in step S8, the normal line determination unit 36 may modify the length of the normal line 108 to be shorter in conjunction with the designation of the reduction, in step S3. For example, the normal line determination unit 36 may shorten the normal line 108 according to a reduction ratio of the rectangular region 106.

The doctor may change the displayed tomographic image 104 by moving the slider 122 using the input device 16A. In this case, the process may return to step S1, and the same process may be performed.

As described above, with the medical image processing device 20 and the medical image processing method, it is possible to reduce the load on the user in a case of designating the three-dimensional region and improve the interpretation accuracy and efficiency.

### Others

The medical image processing device, the medical image processing method, and the medical image processing program according to the present embodiment can also be applied to an image processing device, an image processing method, and an image processing program using a natural image other than the medical image. For example, the medical image processing device, the medical image processing method, and the medical image processing program according to the present embodiment can be applied to a technology for designating a three-dimensional region from three-dimensional image data of social infrastructure facilities such as traffic, electricity, gas, and water supply.

The technical scope of the present invention is not limited to the scope described in the above embodiments. The configurations and the like in the embodiments can be appropriately combined between the embodiments without departing from the gist of the present invention.

### EXPLANATION OF REFERENCES

10: medical image processing system
12: medical image examination device
14: medical image database
16: user terminal device
16A: input device
16B: display
18: interpretation report database
20: medical image processing device
20A: processor
20B: memory
20C: communication interface
22: network
32: image acquisition unit
34: rectangular region acquisition unit
36: normal line determination unit
38: rectangular parallelepiped region determination unit
40: image processing unit
42: output unit
100: three-dimensional image
102-1: tomographic image
102-2: tomographic image
102-i: tomographic image
102-n: tomographic image
104: tomographic image
106: rectangular region
108: normal line
110: three-dimensional region
112: image processing result
112A: image
112B: image
112C: image
114: image processing result
114A: image
114B: image
114C: image
120: slider bar
122: slider
124: range display unit
S1 to S8: step of medical image processing method

What is claimed is:

1. A medical image processing device comprising:
at least one processor; and
at least one memory that stores a command to be executed by the at least one processor,
wherein the at least one processor
displays, on a display device, a first image, which is a tomographic image or cross-sectional image orthogonal to a first direction of three-dimensional medical image data,
receives a rectangular region designated in the displayed first image,
determines a length of a normal line of the first image based on the rectangular region,
determines a three-dimensional region surrounded by a rectangular parallelepiped defined by the rectangular region and the normal line in the three-dimensional medical image data,
performs image processing on the determined three-dimensional region, and
displays, on the display device, a result of the image processing.

2. The medical image processing device according to claim 1,
wherein the at least one processor
receives a modified length of the normal line,
modifies the three-dimensional region with the modified length of the normal line, and
performs the image processing on the modified three-dimensional region.

3. The medical image processing device according to claim 2,
wherein the at least one processor
receives designation of enlargement and/or reduction of the designated rectangular region, and
modifies the length of the normal line in conjunction with the designation of enlargement and/or reduction.

4. The medical image processing device according to claim 1,
wherein the at least one processor
receives the rectangular region having a square shape, and
determines the length of the normal line to be a length of one side of the square shape.

5. The medical image processing device according to claim 1,
wherein the at least one processor
displays the first image and the result of the image processing side by side.

6. The medical image processing device according to claim 5,
wherein the at least one processor
performs the image processing on a past three-dimensional region corresponding to the determined three-dimensional region in past three-dimensional medical image data corresponding to the three-dimensional medical image data, and
displays a result of the image processing on the past three-dimensional region further side by side.

7. The medical image processing device according to claim 1,
wherein the at least one processor
displays, on the display device, a slider bar indicating a position of the first image in the three-dimensional medical image data in the first direction, and
displays, on the slider bar, a range corresponding to the normal line.

8. The medical image processing device according to claim 1,
wherein the at least one processor
displays, on the display device, a numerical value indicating a range corresponding to the normal line in the first direction of the three-dimensional medical image data.

9. The medical image processing device according to claim 1,
wherein the at least one processor
defines a rectangular parallelepiped in which the rectangular region is located at a center in the first direction.

10. The medical image processing device according to claim 1,
wherein the image processing includes three-dimensional reconstruction, and
the result of the image processing includes at least one of an image of an axial cross section, an image of a sagittal cross section, or an image of a coronal cross section.

11. A medical image processing method comprising:
causing at least one processor to:
display, on a display device, a first image, which is a tomographic image or cross-sectional image orthogonal to a first direction of three-dimensional medical image data,
receive a rectangular region designated in the displayed first image,
determine a length of a normal line of the first image based on the rectangular region,
determine a three-dimensional region surrounded by a rectangular parallelepiped defined by the rectangular region and the normal line in the three-dimensional medical image data,
perform image processing on the determined three-dimensional region, and
display, on the display device, a result of the image processing.

12. A non-transitory, computer-readable tangible recording medium on which a program for causing, when read by a computer, at least one processor of the computer to execute the medical image processing method according to claim 11 is recorded.
